(19) United States
(12) Patent Application Publication
(10) Pub. No.: US 2015/0283328 A1
(43) Pub. Date: Oct. 8, 2015

(54) Needle Holder Assembly

(71) Applicants: Thomas J. Shaw, Frisco, TX (US); Retractable Technologies, Inc., Little Elm, TX (US)

(72) Inventors: Thomas J. Shaw, Frisco, TX (US); Mark Small, Heavener, OK (US); Ni Zhu, Plano, TX (US)

(21) Appl. No.: 14/466,722

(22) Filed: Aug. 22, 2014

Related U.S. Application Data
(63) Continuation-in-part of application No. PCT/US13/27485, filed on Feb. 22, 2013.
(60) Provisional application No. 61/601,610, filed on Feb. 22, 2012.

(51) Int. Cl. A61M 5/32; A61M 5/34

(52) U.S. Cl. CPC A61M 5/322; A61M 5/349; A61M 2207/00

(57) ABSTRACT

A needle holder assembly comprising a needle and a needle holder having a longitudinal bore, a bump disposed on the outside of an intermediate portion of the needle prior to assembly with the needle holder, and a needle containment structure disposed inside the bore of the needle holder such that the needle can be inserted rearwardly into the bore until the bump is past the containment structure, after which the bump is not moveable forwardly past the containment structure.

### CROSS-REFERENCE TO RELATED APPLICATIONS

This application is a continuation-in-part of and claims priority to International Application PCT/SU2013/027485, which claims the the benefit of U.S. Provisional Application No. 61/601,610, filed Feb. 22, 2013.

### BACKGROUND

1. Field of the Invention

This invention relates to an improved needle holder assembly for use in medical devices and, more particularly, in medical devices embodying user-activated needle retraction mechanisms.

2. Description of Related Art

Syringes as disclosed for example in U.S. Pat. No. 5,385,551 utilize needle retraction mechanisms comprising a needle holder having a centrally disposed, longitudinal bore with a substantially constant inside diameter. The needle is attached to the walls of the longitudinal bore using glue or other similarly effective means. Because of the small clearances along the sides of the needle, glue sometimes wicks rearwardly and occludes or partially occludes the opening at the blunt, rearwardly facing end of the needle, or is unevenly distributed around the needles so that the needles sometimes detach from the needle holders during use or prior to withdrawal from a patient.

More recently, syringes as disclosed for example in U.S. Pat. No. 5,632,733 utilize needle retraction mechanisms comprising a needle holder having a centrally aligned longitudinal bore with a varying inside diameter. A series of annular recesses is provided in the front portion of the longitudinal bore of the needle holder to provide glue pockets that hold the needle more securely and reduce the likelihood that glue will wick rearwardly around the needle far enough to occlude the rearwardly facing open end of the needle before the glue hardens or is fully cured. Although the use of annular glue pockets near the front of the needle holder is an effective means for securing the needle to the needle holder and for preventing unintentional blockage of the needle, excess or misapplied glue can interfere with needle retraction, and core pins used to mold the needle holders can wear prematurely when withdrawn from the annular recesses, resulting in undesirably higher labor and equipment costs to manufacture the subject needle holders.

### SUMMARY OF THE INVENTION

A needle holder assembly is disclosed that comprises a needle holder with a longitudinal bore into which a needle is inserted, preferably for use in a medical device such as a syringe, a bodily fluid collection device or another vascular access device. The subject assembly further comprises a needle that desirably has a first end insertable rearwardly into the bore from the front of the needle holder and a second end that is pointed and, when installed in the needle holder, projects forwardly from the bore.

According to one embodiment of the invention, the needle is constructed of medical grade material and is provided with at least one “bump,” preferably annular, that is disposed on the outside of the needle shaft in an intermediate position between the first and second ends of the needle. The “bump” is preferably sized and configured to allow passage rearwardly through the bore (upwardly as depicted in FIGS. 1-4) as the first end of the needle is inserted upwardly into the bore. The needle holder is desirably made of a polymeric or glass material, has a substantially cylindrical head and an elongate body projecting forwardly from the head and having an outer diameter smaller than that of the head.

According to one embodiment of the invention, the bore of the needle holder comprises at least one internal or inwardly projecting structure that reduces the inside diameter of the bore at least at the position where it is located.

According to another embodiment of the invention, the bore of the needle holder has a stepped diameter with a smaller diameter front section and a larger diameter rear section, and a rearwardly facing annular shoulder disposed at an interface between the front and rear sections. The length of the needle and bore, and the relative positioning of the “bump” and the rearwardly facing shoulder are desirably such that the pointed end of the needle will project forwardly from the needle holder a desired distance once the needle is installed inside the bore.

According to another embodiment of the invention, the “bump” is an enlarged diameter section of the outer wall of the needle that can be created by any known available means such as by depositing, forming, molding, welding, brazing, soldering, or the like, depending upon the curable or hardenable material from which the “bump” is made.

According to another embodiment of the invention, the “bump” is an enlarged diameter section disposed on the outer wall of the needle that is formed from a cohesive and initially pliable but curable, partially curable or hardenable fixing agent, preferably a material such as glue, adhesive, other polymeric material, or another material including for example and without limitation, a metal or metal alloy. The fixing agent will desirably retain sufficient dimensional stability to remain in place on the needle until cured or hardened, such as by the application of heat or light. Curing, partial curing or hardening of the “bump” can be done prior or after to inserting the needle into the bore of the needle holder, whichever is desired for a particular material and application. Any material used to form the “bump” is desirably cohesive but has a viscosity that allows it to be deposited or formed around the needle shaft at a predetermined axial location along the needle shaft in such manner that the “bump” can be inserted past a rearwardly facing, transverse annular shoulder disposed inside the bore of the needle holder to provide abutting engagement against the shoulder when the glue or adhesive is cured or hardened.

The “bump” will desirably be annular and will frictionally engage the wall of the smaller-diameter section of the bore during needle insertion but can desirably be pushed through the smaller-diameter section of the bore, past the rearwardly facing annular shoulder. Preferably, the cured fixing agent will still retain some degree of resilience that will facilitate insertion of the needle into the needle holder. Once moved past the rearwardly facing shoulder inside the needle holder, the needle should be firmly enough connected to the needle holder that a force greatly in excess of whatever resistance to needle “pull-out” is required for the particular product in which the needle holder assembly is to be used. Alternatively, for use with some needle holder and “bump” configurations and materials, a narrow longitudinal slot or other expansion structure can be provided to permit rearward passage of a bump through a front portion of the bore and past a retainer structure that will limit subsequent forward movement of the “bump” relative to the front portion of the bore.

According to another embodiment of the invention, the “bump” is made with, or will be resilient enough to form, a forwardly facing annular shoulder that provides facing engagement with the rearwardly facing annular shoulder in the bore of the needle holder after the needle is installed inside the needle holder.

### BRIEF DESCRIPTION OF THE DRAWINGS

The needle holder assembly of the invention is further described and explained in relation to the following figures of the drawings, in which:

FIG. 1 is an exploded, cross-sectional front elevation view of the needle holder and needle of one embodiment of the invention, with a quantity of fixing agent deposited on the needle shaft;

FIG. 2 is a cross-sectional front elevation view of the needle holder of the invention as assembled for use in a medical device having a retractable needle assembly;

FIG. 3 is an enlarged, detail view of the needle of FIG. 1; and

FIG. 4 is an enlarged, detail view of the upper portion of the needle holder of FIG. 2.

Like reference numerals are used to describe like parts in all figures of the drawings.

### DESCRIPTION OF A PREFERRED EMBODIMENT

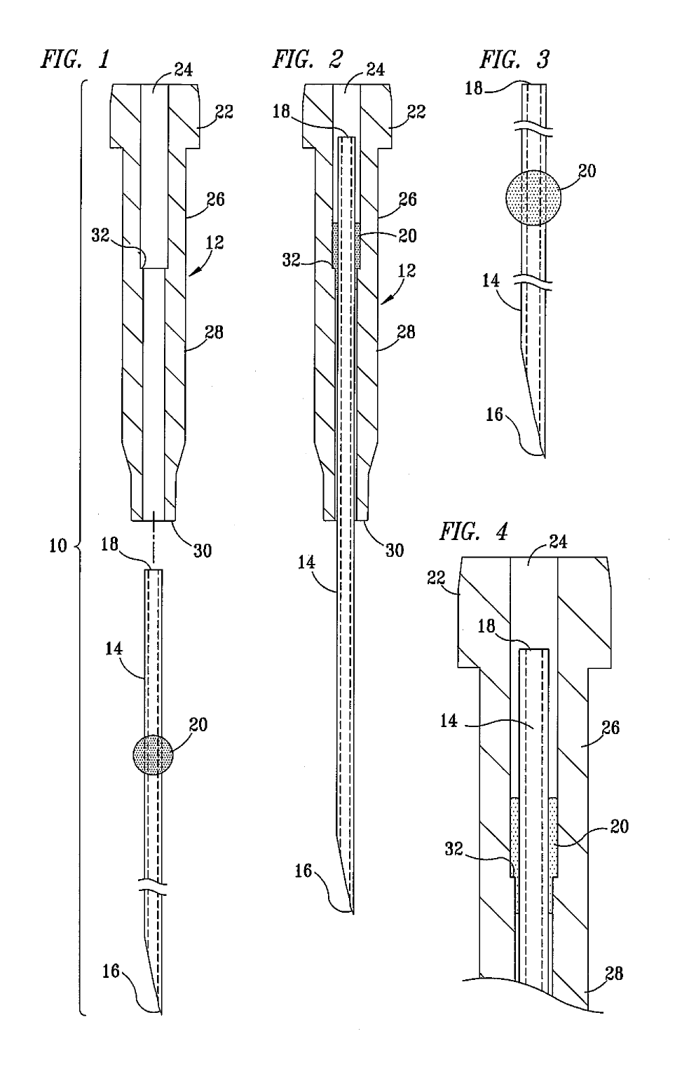

Referring to FIGS. 1-4, needle holder assembly 10 of the invention desirably comprises needle holder 12, needle 14, and a “bump” comprising a controlled quantity of fixing agent such as adhesive 20 or other suitable similarly effective curable or hardenable material as disclosed above having a viscosity low enough to render it sufficiently pliable to be inserted through the smaller diameter section of bore 24 of front part 28 of elongate body portion 26 but high enough that it will not flow rearwardly to occlude rear opening 18 of needle 14. The “bump” is desirably formed on needle 14 prior to inserting needle 14 into needle holder 12. Depending upon the configuration of the needle holder 12 and needle 14, and the adhesive 20 or other curable or hardenable material chosen to create the “bump” on needle 14, curing can be done either before or after needle 14 is positioned inside needle holder 12.

Needle holder 12 is preferably made of a moldable polymeric material and further comprises head 22, and elongate body portion 26 with an outer diameter less than that of head 22. The end of needle 14 with rear opening 18 is desirably insertable into needle holder 30 at front end 30 after a quantity of adhesive 20 is deposited at a predetermined location on needle 14 that corresponds to the position of rearwardly facing, transverse annular shoulder 32 of needle holder 12. After the quantity of adhesive 20 is in place and cured or hardened by conventional means, it desirably abuts against the rearwardly facing annular shoulder 32 to provide additional holding force to needle 14 relative to needle holder 12 to prevent needle 14 from being separated from needle holder 12 during use, and particularly, prior to needle retraction, when beveled front tip 16 of needle 14 is still disposed inside a patient.

Alternatively, adhesive 20 or another curable or hardenable material can be cured, partially cured or hardened prior to insertion of needle 14 into bore 24 of needle holder 12. Also, a plurality of inwardly facing, circumferentially spaced projections restricting bore 24 of needle holder 12 can be provided in place of the stepped internal bore 24 having a rearwardly facing annular shoulder 32 if desired.

Other alterations and modifications of the invention will likewise become apparent to those of ordinary skill in the art upon reading this specification in view of the accompanying drawings, and it is intended that the scope of the invention disclosed herein be limited only by the broadest interpretation of the appended claims to which the inventors are legally entitled.

1. A needle holder assembly comprising a needle holder and a needle, wherein:
the needle holder comprises a longitudinal bore having a front portion, a rear portion, an inside diameter, and at least one retaining structure disposed between the front and rear portions; and
the needle comprises a first end disposed inside the bore and a second end projecting forwardly from the bore, and a quantity of cured or hardened fixing agent forming a bump on an outer wall of the needle between the first end and the second end; and
wherein the bump is formed on the needle prior to assembly of the needle and needle holder and is configured to be insertable through the front portion and past the retaining structure during assembly of the needle and needle holder to limit subsequent forward motion of the needle in relation to the needle holder.

2. The needle holder assembly of claim 1 wherein the needle holder has a stepped inside bore with a smaller diameter front portion and a larger diameter rear portion.

3. The needle holder assembly of claim 2 wherein the inwardly facing structure is a rearwardly facing annular surface disposed between the front portion and the rear portion.

4. The needle holder assembly of claim 1 wherein the fixing agent is a curable or hardenable material.

5. The needle holder assembly of claim 4 wherein the cured or hardened fixing agent is selected from the group consisting of glue, adhesive, other polymeric materials, metal and metal alloys.

6. The needle holder assembly of claim 1 wherein the bump is cured or hardened before the needle is inserted into the needle holder.

7. The needle holder assembly of claim 1 wherein the bump is cured or hardened after the needle is inserted into the needle holder.

8. The needle holder assembly of claim 1 wherein the fixing agent is applied by an application technique selected from the group consisting of forming, depositing, molding, welding, brazing, soldering, and combinations thereof.

9. The needle holder assembly of claim 3 wherein the bump has a forwardly facing shoulder that abuts against the annular surface.

10. A method for making a needle holder assembly comprising a needle and a needle holder having a longitudinal bore, the method comprising:
providing on the outside of the needle in a position intermediate to a front end and a rear end of the needle a bump that effectively increases an outside diameter of the needle at the position;
providing a needle containment structure inside the bore of the needle holder;
inserting the rear end of the needle into a front end of the bore; and
sliding the needle rearwardly inside the bore until the bump is disposed rearwardly of the needle containment structure.
